(19) United States
(12) Patent Application Publication
(10) Pub. No.: US 2006/0184189 A1
(43) Pub. Date: Aug. 17, 2006

(54) Cap for a dermal tissue lancing device

(76) Inventors: Lorin Olson, Scotts Valley, CA (US); Anne Thomson, Strathpeffer (GB); Damian Edward Haydon Baskeyfield, Inverness (GB); Christopher Philip Leach, Inverness (GB); Richard Michael Day, Cawdor, Naim (GB); Sebastian Bohm, Inverness (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/706,166

(22) Filed: Nov. 12, 2003

Related U.S. Application Data
(60) Provisional application No. 60/426,683, filed on Nov. 15, 2002.

(51) Int. Cl. A61B 17/32

(52) U.S. Cl. 606/181

(57) ABSTRACT

A cap for use with a dermal tissue lancing device (that includes a housing and a lancet movable with respect to the housing) includes a proximal end for engaging with the housing, a distal end for engaging with dermal tissue and an opening for a portion of the lancet to pass through. The distal end of the cap includes portions for engaging dermal tissue. These portions are resiliently deformable such that, when the cap contacts and is urged towards dermal tissue, the portions deform resiliently, engaging the dermal tissue and approaching theretogether. A process for the collection of a fluid sample (e.g., blood) from dermal tissue includes providing a dermal tissue lancing device with a housing, a cap and a lancet that is moveable with respect to the housing and the cap. The cap includes a proximal end for engaging with the housing, a distal end for engaging with dermal tissue, and an opening for a portion of the lancet to pass through. The distal end of the cap includes resiliently deformable portions for engaging dermal tissue. Next, the cap is contacted with the dermal tissue (e.g., dermal tissue of a fingertip, limb, or abdomen) such that the portions engage the dermal tissue. Subsequently, the cap is urged towards the dermal tissue such that at least first and second portions deform resiliently and approach theretogether to create a bulge in the dermal tissue. The bulge is then lanced to create a puncture from which a fluid sample is collected.

### BACKGROUND OF THE INVENTION

1. Field of the Invention

The present invention relates, in general, to dermal tissue lancing devices and, in particular, to caps for dermal tissue lancing devices and associated methods.

2. Description of the Related Art

Lancets in conventional use generally have a rigid body and a sterile needle that can be cocked and fired so as to briefly protrude from one end of the lancet. In a conventional lancing device, a lancet is mounted within a longitudinal housing and is movable along a longitudinal axis relative to the housing. Typically the lancet is spring loaded and driven along the longitudinal axis on release of the spring to puncture (i.e., “lance” or cut) dermal tissue. A blood sample can then be expressed from the punctured dermal tissue by squeezing (i.e., “milking”) the finger, or other area of the body, that has been punctured for sample collection.

The lancet is used to pierce dermal tissue, thereby enabling the production of a fluid sample, typically blood, from the puncture and collection of the fluid sample for testing for an analyte, such as glucose. The blood is then transferred to a test collection device (e.g., a test strip). This test collection device may be part of a completely separate sample collection and metering system or it may form part of a combined lancet and metering system, such as that disclosed in International Application No. PCT/GB02/03772 (published as WO 03/015627 on Feb. 27, 2003), which is hereby fully incorporated by reference.

Blood samples are most commonly taken from the fingertips, where there is generally an abundant supply due to the presence of capillary blood vessels. However, the nerve density in the fingertips can cause significant pain in many patients. Sampling on alternative sites, such as earlobes, palms, limbs and the abdomen, is sometime practiced since these alternative sites may be less sensitive. However, these alternative sites are also less likely than a fingertip to provide sufficient blood volume. Use of these alternative sites also makes blood transfer directly to test devices difficult, particularly when a combined lancet and metering device is employed.

After puncturing dermal tissue, conventional lancing devices are put to one side and the user squeezes blood from the puncture wound. This technique requires a clean storage site for the lancing device and a two-handed, two-step operation. Once a drop of blood is expressed from the lancing site, the user transfers the blood to a test strip or suitable meter.

Conventional lancing devices are available from, for example, LifeScan, Inc. of Milpitas, Calif., Palco Laboratories of Santa Cruz, Calif., Therasense of Alameda, Calif. and Amira Medical of Scotts Valley, Calif. Conventional lancing devices are described in U.S. Pat. No. 5,730,753 to Morita, U.S. Pat. No. 6,045,567 to Taylor et al. and U.S. Pat. No. 6,071,250 to Douglas et al., each of which is incorporated fully herein by reference.

Furthermore, typical lancing devices often include a cap that engages the dermal tissue and through which the lancet protrudes on firing. The cap will, therefore, have an aperture (i.e., opening), through which the lancet will pass on firing. Typically, a distal end of the cap will be placed in contact with the dermal tissue immediately prior to firing.

When a dermal tissue lancing device with a conventional cap is placed in contact with dermal tissue, a small amount of pressure is typically applied by a user prior to launch of the lancet. This pressure forces the cap down upon the dermal tissue in a direction generally perpendicular to the surface of the dermal tissue. A small amount of dermal tissue can pass through the aperture and form a bulge, into which the lancet is launched and a puncture formed. Nevertheless, typically no blood is visible on removal of the cap and lancing device from the dermal tissue. In order to produce a blood drop that is large enough for introduction onto a test strip and subsequent measurement by a metering device, the area surrounding the puncture must be squeezed by the user.

Obtaining a blood sample in excess of 0.5 μl using a lancing needle without subsequent manipulation of the skin adjacent to the lanced cut can be problematic. To obtain larger amounts of blood, pressure (such as a pumping or milking action) is usually applied to the skin adjacent to the lanced cut, in order to force additional blood out through the cut. Some devices combine lancing and transfer to a test cell of the blood produced on lancing into an integral unit without repositioning the device. One such device is a blood glucose measuring meter that is positioned on the dermal tissue over the site to be tested. These devices do not expose the test site adequately for efficient pumping action. Vacuum at the cut has been used but is difficult to implement reliably.

This means that a user has to lay aside the metering device to squeeze the puncture area and produce blood before picking up a metering device and placing this in contact with the newly formed drop of blood.

Still needed in the field, therefore, is a cap for a dermal tissue lancing device that enables a user to obtain a fluid sample (e.g., a blood sample) without subsequent manipulation (e.g., squeezing and/or milking) of a lanced area. In addition, the cap should be compatible with use on a variety of testing sites (e.g., fingertips, limbs and abdomen).

### SUMMARY OF THE INVENTION

Embodiments of the present invention include a cap for use with a dermal tissue lancing device that enables a user to obtain a fluid sample (e.g., a blood sample) without such subsequent manipulation as squeezing and/or milking of a lanced area. Caps in accordance with embodiments of the present invention are compatible with use on a variety of testing sites (e.g., fingertips, limbs and abdomen). Other embodiments of the present invention include methods for collecting a fluid sample using a dermal tissue lancing device that do not require such manipulation of squeezing and/or milking of a lanced area subsequent to a lancing step.

A cap according to an exemplary embodiment of the present invention is adapted for use with a dermal tissue lancing device that includes a housing and a lancet. The lancet is movable with respect to the housing. The cap includes a proximal end for engaging with the housing, a distal end for engaging with dermal tissue and an opening (i.e., aperture) for a portion of the lancet to pass through. The distal end of the cap includes at least first and second portions for engaging dermal tissue. These first and second portions are resiliently deformable such that, when the cap contacts and is urged towards dermal tissue, the portions deform resiliently to engage the dermal tissue and approach theretogether.

A method for the collection of a fluid sample (e.g., a blood sample) from dermal tissue according to an exemplary embodiment of the present invention includes first providing a dermal tissue lancing device. The dermal tissue lancing device thus provided includes a housing, a lancet that is moveable with respect to the housing and a cap. The cap itself includes a proximal end for engaging with the housing, a distal end for engaging with dermal tissue and an opening (i.e., aperture) for a portion of the lancet to pass through. Furthermore, the distal end of the cap includes at least first and second resiliently deformable portions for engaging dermal tissue.

The cap of the dermal tissue lancing device is then contacted with the dermal tissue (e.g., dermal tissue of a fingertip, limb, abdomen or other site from which a fluid sample is to be collected) such that the at least first and second portions engage the dermal tissue. Subsequently, the cap is urged towards the dermal tissue (using, for example, a predetermined force) such that the at least first and second portions deform resiliently and approach theretogether. Such an approach creates a bulge in the dermal tissue by, for example, decreasing the size of the dermal tissue lancing device opening. The bulge is then lanced, using the lancet, to create a puncture in the bulge, from which a fluid sample is collected.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings in which like numerals indicate like elements, objects and forces, of which:

FIG. 1A is a perspective view of a cap for use with a dermal tissue lancing device according to an exemplary embodiment of the present invention;

FIG. 1B is a bottom (end) view of the cap of FIG. 1A;

FIG. 2A is a perspective, partially-cut-away view of the cap body illustrated in FIG. A in a relaxed state;

FIG. 2B is a perspective view of the cap body illustrated in FIG. 1A in a relaxed state;

FIG. 2C is a perspective, partially-cut-away view of the cap body of FIG. 1A in a compressed state as can be encountered during use (for example, when a user urges a lancet device or combined lancet and metering device incorporating such a cap against skin);

FIG. 2D is a perspective view of the cap body of FIG. 1A when compressed during use;

FIG. 3 is a perspective, partially-cut-away view of the cap body of FIG. 1A in use compressing dermal tissue illustrating a bulge 32 formed in the dermal tissue 33 under pressure applied by a user in direction 40;

FIG. 4 is a perspective, partially cut away view of the cap of FIG. 1;

FIG. 5A is a perspective view of another exemplary embodiment of a cap and retaining ring according to the present invention;

FIG. 5B is an end view of the cap of FIG. 5A;

FIG. 6A is a perspective partially cut away view of the cap body of FIG. 5A, in a relaxed state prior to use;

FIG. 6B is a perspective view of the cap body of FIG. 5A in a relaxed state prior to use;

FIGS. 6C and 6D are perspective (and for FIG. 6C partially cut away) views of the cap body of FIG. 5A when partially or wholly compressed against the dermal tissue of a user during lancing;

FIG. 7 is a perspective, partially cut away view of the cap body of FIG. 5A when compressed against the dermal tissue of a user illustrating the direction of pressure (40) applied by a user and a bulge 132 formed in the dermal tissue;

FIG. 8 is a perspective partially cut away view of the cap of FIG. 5A;

FIGS. 9A and 9B illustrate respectively the cap of FIG. 5A immediately prior to compression against the dermal tissue and immediately after compression against the dermal tissue and launch of the lance (Lancet 150—not to scale);

FIGS. 10A and 10B show respectively, uncompressed and compressed cross-sectional views of the cap body of FIG. 1A;

FIGS. 11A and 11B show, respectively, uncompressed and compressed (or collapsed) cross-sectional views of the cap body of FIG. 5A according to the invention;

FIG. 11C is a cross-sectional view of the cap of FIGS. 5A, 11A and 11B when compressed ready for launch of the lancet (not shown), with FIG. 11C1 being an exploded view of a portion encircled by the dashed arrows in FIG. 11C;

FIGS. 12A and 12B are schematic, cross-sectional views of a conventional lancing device and a lancing device according to the present invention incorporating the cap of FIG. 1A, respectively;

FIG. 13 is a flow diagram illustrating a sequence of steps in a process according to one exemplary embodiment of the present invention;

FIGS. 14A through 14E are schematic cross-sectional views a lancing device in accordance with the present invention incorporating the cap of FIG. 5A and illustrating multiple lancet positions;

FIG. 15 is a perspective cross-sectional view of a lancing device according to an embodiment of the present invention that includes a cap that is also according to an embodiment of the present invention;

FIG. 16A is a simplified perspective view of a cap according to a further embodiment of the present invention;

FIGS. 16B and 16C show, respectively, initial and compressed states of the cap of FIG. 16A.

FIG. 16D depicts a manner in which a plurality of the caps of FIG. 16A can be stacked; and

FIGS. 17A and 17B depict, in cross-section, a cap according to the present invention prior to, and subsequent to, compression between two rigid surfaces.

### DETAILED DESCRIPTION OF PREFERRED EMBODIMENTS

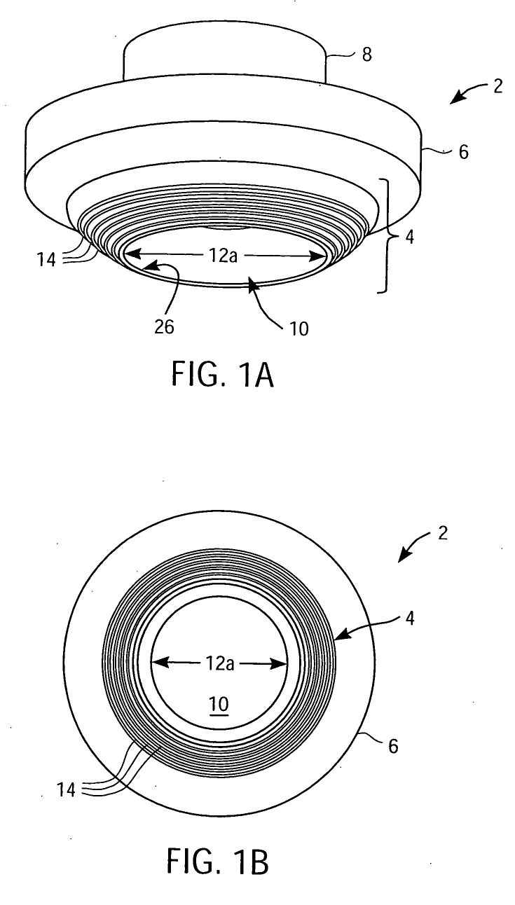

FIGS. 1A and 1B illustrate perspective and bottom (end-on) views, respectively, of a cap 2 for use with a dermal tissue lancing device (not shown) according to an exemplary embodiment of the present invention. In the embodiment of FIGS. 1A and 1B, cap 2 includes a cap body 4 (such as a flexible cap body) and retainer 6. Once apprised of the present disclosure, however, one of ordinary skill in the art can envisage alternative cap embodiments in which retainer 6 is not present. For example, a cap body of varying resilience (e.g., either of a stepped or a graded resilience) having (i) a distal end for engaging dermal tissue composed of relatively resiliently deformable material and (ii) a proximal end for engaging with a dermal tissue lancing device composed of a relatively less resiliently deformable material can be employed without a retainer.

Cap body 4 includes an opening 10 (that is defined by inner edge 26 of cap body 4) and dermal tissue engaging features 14. Retainer 6 includes a stem 8. Other features of cap body 4 and retainer 6 are described below in conjunction with FIGS. 1A, 1B, 2A through 2D, 3 and 4.

Cap 2 is configured to facilitate the flow of a bodily fluid sample (e.g., a blood sample) out of a lancet cut (e.g., a puncture) in dermal tissue without such manipulation of squeezing and/or milking of the dermal tissue subsequent to lancing. In the embodiment of FIGS. 1A and 1B, cap body 4 is formed of an elastomeric material. During use, cap body 4 encircles an area of the dermal tissue that is to be lanced through opening 10 (also referred to as an aperture) in the cap body. The diameter of opening 10 when cap body 4 is in a relaxed state is denoted as distance 12a in FIG. 1A.

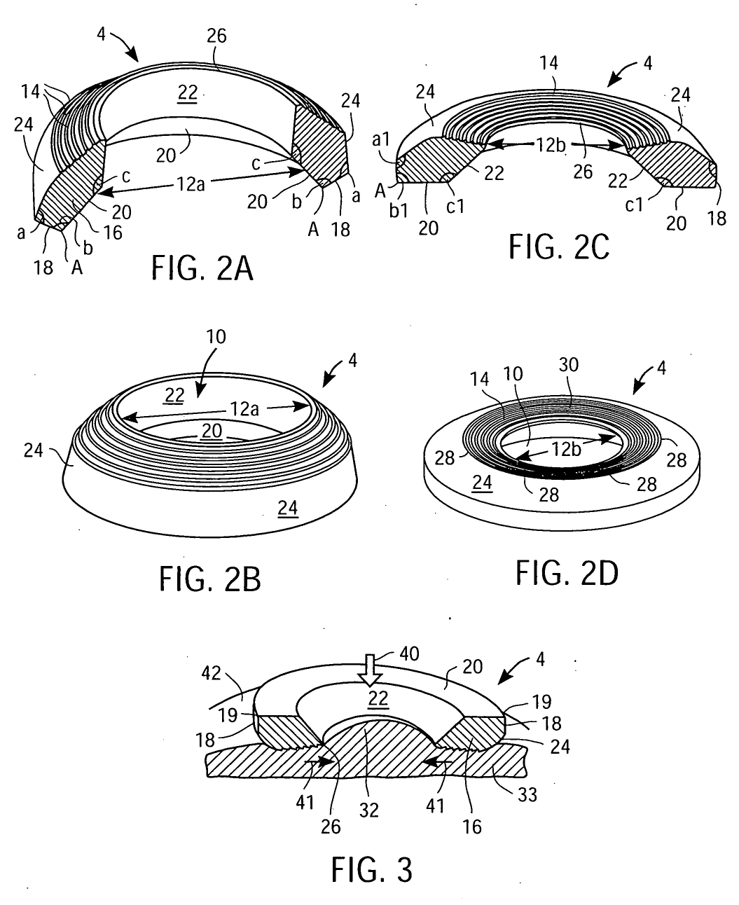

In the embodiment of FIGS. 1A and 1B, inner edge 26 defines a circular opening 10, however, the shape of opening 10 can be any suitable shape including, but not limited to, a square shape, a triangular shape, a C-shape, a U-shape (thus allowing access to opening 10 from the side by, for example, a test strip to enable in-situ transfer of a blood sample to the test strip), an hexagonal shape and an octagonal shape. Furthermore, cap body 4 can have a plurality of resiliently deformable segments (i.e., portions), not limiting to two, the distal ends of which engage the skin and, on compression, these segments can resiliently deform urging the dermal tissue into a bulge. Preferably, but not necessarily, these segments resiliently deform and approach theretogether in a synchronized way to create a bulge in the dermal tissue. The embodiment of FIGS. 1A and 2A, however, has an opening 10 with a continuous inner edge 26 through which skin can pass, as well as dermal tissue engaging features 14 for engaging the skin and urging it into a bulge upon compression of cap body 4 of cap 2.

Retainer 6 includes an optional stem 8 for connecting cap 2 to a housing (not shown) of a dermal tissue lancing device. Stem 8 may, if desired, be threaded to mate with a corresponding thread on the housing. Alternative connecting mechanisms may be used, including, but not limited to, a snap fit connection and a telescoping connection. Indeed, stem 8 may be fixably connected to the housing, although a degree of controlled movement of cap 2 with respect to the housing along the direction of lancing will allow control of the position of the opening 10 in cap body 4 with respect to the lancet in a rest position.

In the embodiment of FIGS. 1A and 1B, cap body 4 is formed of an elastomeric material and is readily resiliently deformable for insertion into retainer 6 and use. Thus, a user can squeeze sides (e.g., base surface 18) of the cap body together and slip the thus deformed cap body into the retainer, whereafter the cap body returns to an essentially undeformed shape and is ready for use. In the embodiment of FIGS. 1A and 1B, cap body 4 is rotatably snug fit within retainer 6 (as discussed in detail below).

Cap body 4 includes dermal tissue engaging features 14 in the form of a plurality of concentric protruding ridges that surround opening 10. Alternative dermal tissue engaging features, such as a roughened surface, other forms of protrusion, recesses, etc., can be envisaged by those skilled in the art once they are apprised of the present disclosure.

FIGS. 2A and 2B depict a cross-sectional perspective and perspective views, respectively, of cap body 4 in an initial relaxed, non-deformed, uncompressed state. Alternatively, embodiments can be envisaged by those skilled in the art in which the initial (i.e., prior to use) state of the cap 2 is partially compressed (e.g., partially compressed within the confines of retainer 6). A cross-section 16 through cap body 4 is, for example, generally squat, and thus the height of the cross-section is generally of the same order of dimension as the width of the cross-section. The distal end of cylindrically shaped internal surface 22 of cap body 4 defines inner edge 26 of opening 10. A second internal surface 20 of cap body 4 is frusto-conical in shape and adjoins internal surface 22 at its proximal end and base surface 18 of cap 4 at its distal end. Base surface 18 meets outer surface 24, which is of an approximately cylindrical shape.

Opening 10 of cap 2 is typically circular. Alternative suitable opening shapes, such as square, triangular, rectangular, hexagonal, octagonal or any suitable shape for engaging skin can be envisaged by those skilled in the art, as can the use of a cap that includes separate segments (i.e., separate portions) or fingers, the ends of which essentially define at least a part of the opening and that resiliently deform under pressure to urge the formation of a target site bulge for lancing within the opening.

FIGS. 2C and 2D illustrate cap body 4 in an activated (i.e., compressed) state. Once cap body 4 is partially or wholly compressed (due to, for example, contact with and urging against dermal tissue [i.e., skin] during lancing use), the diameter 12b of opening 10, that is a reduced diameter relative to the diameter 12a of cap body 4 in a relaxed or partially relaxed state, is obtained. Similarly the diameter of dermal tissue engaging features 14 can be reduced on compression of cap body 4.

Referring to FIGS. 1A, 1B, 2A through 2D, 3 and 4, during lancing, a user presses the lancing device and, hence, cap 2, onto the skin (i.e., onto a dermal tissue target site, also referred to as a testing site). Such pressing applies a force to resiliently deformable cap body 4 that is substantially perpendicular to the dermal tissue (i.e., to a plane containing opening 10), resulting in opening 10 being adjacent with the surface of the skin. This force deforms (e.g., compresses) cap body 4, causing inner edge 26 and dermal tissue engaging features 14 to move radially inwards, thus reducing the size of the opening to dimension 12b. A bulge is formed in the skin encircled by the reduced opening and, following lancing of the bulge, bodily fluid (e.g., blood) emerges from the puncture without such manipulation as squeezing and/or milking of the lanced area. To increase the amount of blood produced, the application of the force on the skin by the lancet device/cap can be maintained for a predetermined time period after lance (i.e., post-lance pressure). The longer the post-lance pressure time period, the more blood is generally produced. The amount of blood produced can be further increased by also applying and maintaining such force prior to lance (i.e., pre-lance pressure) for a predetermined time period. One skilled in the art can optimize production of blood by experimenting various combination of pre-lance pressure and/or post-lance pressure time duration. Preferred post-lance pressure time duration for producing sufficient amount of blood to be used for analyte testing (e.g., glucose concentration testing) is approximately 5 seconds. Caps according to the present invention are particularly useful in combined lancet and metering devices for measuring an analyte such as glucose. More particularly, caps according to the present invention are useful for such devices which measure an analyte in-situ, since there is no need to reposition the device, following lancing, for squeezing and/or milking the lanced area for production of a sample.

Movement of inner edge 26 and dermal tissue engaging features 14 radially inwards causes skin, blood and sub-dermal tissue beneath the skin into a bulge within the circumference of the opening (i.e., aperture). Such movement is speculated to result in an increased (relatively high) pressure within the bulge.

On compression, cap body 4 pivots inwards about circular ridge point A so that second internal surface 20 becomes a base (i.e., the proximal end of cap body 4 closest to the lancing device). Second internal surface 20 changes from a frusto-conical shape into a disc-like shape. Likewise, internal surface 22 loses its cylindrical shape and becomes frusto-conical in shape. The diameter 12b of opening 10 reduces compared to diameter 12a (i.e., portions of inner edge 26 and dermal tissue engaging features 14 approach one another as the opening reduces in size).

It is speculated without being bound that the following discussion provides an explanation of the effect of caps according to the present invention on lancing. The force applied by a user in a direction generally perpendicular to a plane of opening 10 is translated into a force acting radially inwardly on the dermal tissue by the action of the retainer 6 on the cap body 4. This can be seen more clearly in FIG. 3, in which the force in direction 40 (indicated by the open arrow of FIG. 3) causes radially inwardly directed force 41 (indicated by the closed arrows in FIG. 3) to act upon the dermal tissue, thereby forming the dermal tissue into a bulge 32.

Typically, force 41 can result from the reaction of the cap body 4 acting radially outward on the retainer 6 during its resilient deformation. The term “resiliently deformable” means that on release from an external force (e.g., force 41), the cap body at least partially and in some embodiments wholly resumes its original shape (optionally within the confines of a retainer in the circumstance that such a retainer is provided). For the purpose of explanation only, retainer 6 is illustrated as circular and continuous in FIGS. 1A and 1B. One skilled in the art will recognize that retainer 6 may be of any suitable shape or configuration, including but is not limited to, square, triangular and hexagonal, or it may be formed as discrete segments defining such shapes. Sub-dermal material and blood are also drawn into the bulge. Surface tension in the dermal tissue within the bulge 32 imparts pressure to the bulge. In addition, the provision of an inner edge 26 provides a pinch point, or more precisely a pinch ring, at inner edge 26, thereby providing a local increase in pressure and restricting blood flow out of bulge 32.

The provision of a distinct sharply curved inwardly facing inner edge 26 on opening 10 is speculated to enable pressure from the resiliently deformed cap to be exerted directly onto the skin that has passed through the opening. This urges the formation of a bulge and constricting of the dermal tissue as blood and sub-dermal tissue are forced into the bulge whilst limiting return slippage of the skin past the internal edge. Furthermore, once blood and sub-dermal tissue are forced into the bulge within the opening, portions of the inner edge are believed to provide a higher pressure region and/or pinch points limiting the ability of the blood and sub-dermal tissue below the skin to exit the bulge despite the exertion of pressure to do so from surface tension caused by the constriction of the skin forming the bulge. It is further speculated without being bound that compression of the cap body 4 occurs in two stages. In an initial stage, the cap body 4 serves to apply a ring of pressure approximately perpendicular to the skin surface that constricts the flow of blood into and out of the ringed area. In a second stage, the diameter of the inner ring in contact with the skin decreases so that blood that is already constrained by the cap body is brought further into the center of the ringed area and compressed.

Bulge 32 can be centered within the target area of the lancing needle. Furthermore, due to the presence of a bulge, when a lancet (e.g., a needle-type lancet) punctures the dermal tissue, the resulting blood flow is larger than from the lanced cut made by a conventional lancing device without using caps according to embodiments of the present invention.

Materials other than elastomeric materials can be used to form a cap according to embodiments of the present invention as long as, for example, a force essentially perpendicular to the dermal tissue results in deflection of the material and hence deflection of the skin (i.e., dermal tissue) in such a way as to force the blood beneath the skin into a bulge. For example, a cap body 4 can be made of deformable polystyrene. Thus, on initial compression, such a cap would compress the skin to form a bulge as herein described, but further compression could result in permanent deformation of the cap body. Thus the cap body is prevented from being re-used more than once. This can be an advantage by reducing the risk of infection from a cap becoming contaminated with body fluid during a first lancing operation and then being re-used for a later lancing operation. The following description is given by way of speculative explanation but is not intended to be limiting.

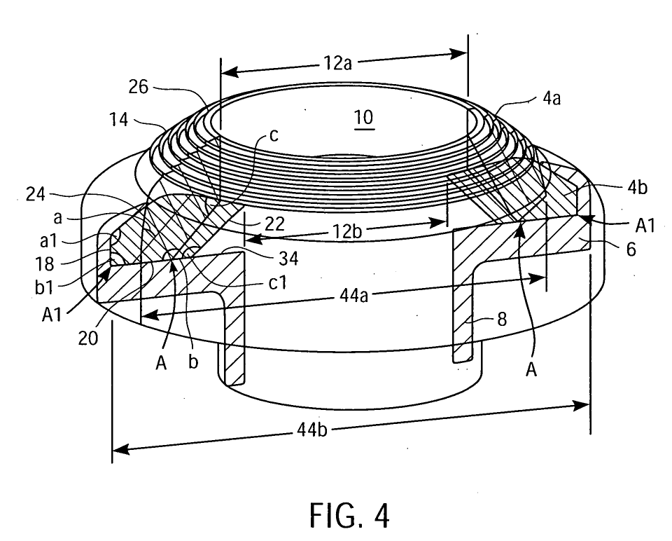

Rotation or pivoting of cap body 4 can be seen in FIG. 4 in which cap body 4a is in a relaxed state and cap body 4b is shown in a compressed state. On compression of cap body 4a into position 4b, angles a, b and c become angles a1, b1 and c1, respectively. In addition, pivot A not only acts as a pivot, but also moves radially outwards to form an outer corner of newly formed disc-shaped second internal surface 20. Thus, the region of the cap body immediately outside the opening is pushed toward the center of the opening pulling the dermal tissue within it. The external diameter of the base region of cap body 4 expands from 44a in relaxed state to 44b in a compressed state.

It can be seen in this diagram that diameter 12a of opening 10 in a relaxed state is greater than diameter 12b of opening 10 in a compressed state. It can also be seen in FIG. 4 that when cap body 4 is in a relaxed state, it is free to rotate within a recess provided in retainer 6. This means that cap body 4 can be readily inserted into retainer 6 ready for use, or removed for cleaning, by simply deforming (e.g., by pinching) and sliding cap body 4 in and out of retainer 6.

A stop surface 34 (also referred to simply as a “stop”) is provided to limit the compression of cap body 4 in a longitudinal direction (i.e., generally perpendicular to a plane containing opening 10) with respect to the lancing device. Typically the lance also extends and retracts along this direction, although it may act at an angle to this direction.

Within a lancing device, the use of a stop enables a lancet rest and cocked position to be determined with respect to the stop and therefore with respect to a bulge of dermal tissue of nominal size protruding within the opening. Therefore, it is possible to determine or estimate for a nominal bulge size the depth a lancet will penetrate on launch, as long as the cap body is deformed sufficiently to have reached the stop position. Thus, by controlling the location of the lance with respect to the stop and/or the cap with respect to the stop, the depth of penetration of the lance into the dermal tissue can be controlled. By trial and error, the user will be able to determine for each particular location about the body what settings of lance with respect to stop and/or cap with respect to stop is required to produce a suitably sized drop of blood with the least or an acceptable amount of discomfort.

Furthermore, it will be appreciated by those skilled in the art that as a user pushes (presses) cap body 4 down onto the dermal tissue, the base region of cap body 4 (closest to the lancing device) will travel radially outwards and could be limited by the inner surface of retainer 6. Thus, it is speculated without being bound that retainer 6 may provide a reaction force so that further compression of cap body 4 causes radially inward travel of inner edge 26 of opening 10, rather than radially outward travel of pivoting ring A. This particular form of cap body is especially suitable for relatively hard dermal tissue such as the fingers, although it can be used elsewhere on a body.

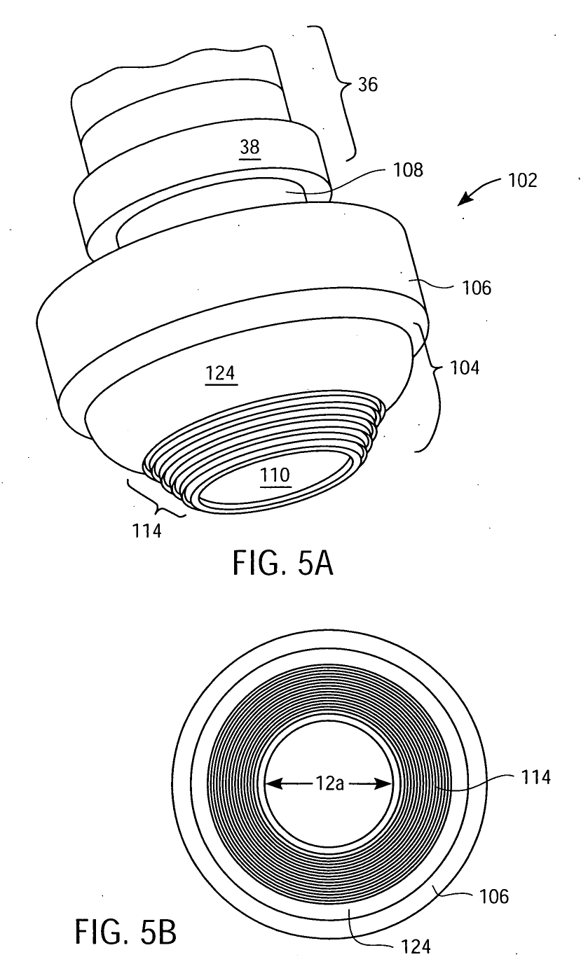

FIGS. 5A and 5B illustrate perspective and end views, respectively, of an alternative embodiment of a cap according to the present invention. Here, cap 102 includes a cap body 104 and a retainer 106. Retainer 106 is mounted to a lancing device by a stem 108, mount 36 and a connecting ring 38. Cap body 104 includes an outer surface 124, and an opening 110 and a series of dermal tissue engaging features 114 in the form of concentric protruding ridges.

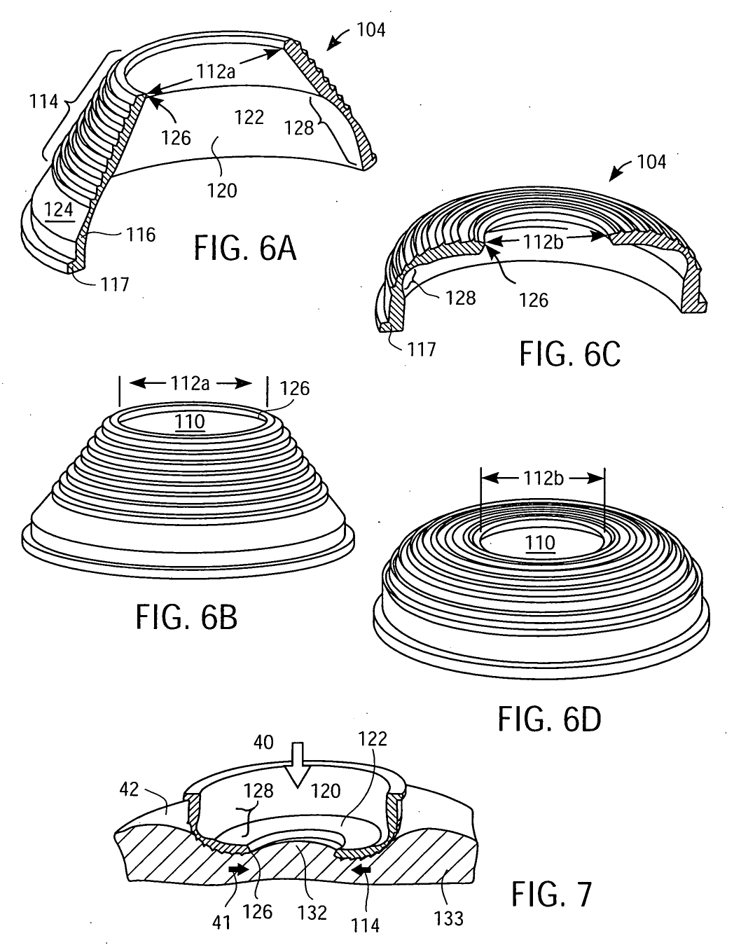

FIGS. 6A-6D illustrate perspective and perspective cross-sectional views of cap body 104 in relaxed (uncompressed) and activated (compressed) states. The cross-sectional shape 116 of cap body 104 is different from that of cap body 4. The cross-section is elongate such that cap body 104 is generally frusto-conical in shape having two inner facing surface portions 120 and 122 that meet one another at an area of weakness 128. Area of weakness 128 may be provided by a thinning of the cross-section of cap body 104 in this region. Thus a cap body with a ring of variable cross-section may be provided in one exemplary embodiment. Such a variable cross-section provides one or more areas of weakness (e.g., in a ring concentric within the cap itself) to facilitate cap collapse and the formation of a skin bulge. Inner edge 126 defines opening diameter 112a (when in an relatively uncompressed state) and opening diameter 112b (when in a relatively compressed state). It should be noted that opening diameter 112b is less than opening diameter 112a.

A lip 117 is fixably mounted to retainer 106, as shown in FIGS. 6A and 6C. Referring now also to FIG. 7, it is speculated, without being bound, that when a predetermined force is applied in direction 40 (i.e., towards dermal tissue 42) by a user urging the lancing device and cap body 104, a force 41 is generated acting radially inwards since lip 117 is prevented from moving radially outwards. Cap body 104 then folds in on itself about weakness 128 and a force 41 directed radially inwards is generated within the dermal tissue surface causing the formation of a bulge 132. Sub-dermal tissue 133 and blood are urged into bulge 132 and, to a certain extent, prevented from leaving by the action of inner edge 126, thereby maintaining a relatively high pressure within bulge 132. Lancing of such a bulge produces a relatively high amount of blood.

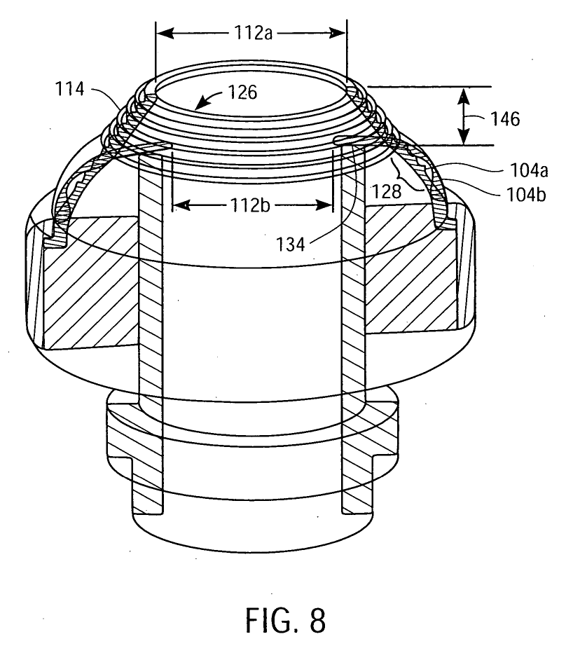

FIG. 8 shows in more detail the change in shape of cap body 104 on compression (from 104a to 104b). A stop 134 limits the decrease in height of the cap body 104 to a distance 146. Cap body 104 includes an area of weakness 128 about which folding can take place. In the embodiment of FIG. 8, the area of weakness 128 is provided by a narrowing of the cross-section of cap body 104 in this region. As cap body 104 folds over, opening diameter 112a decreases to diameter 112b.

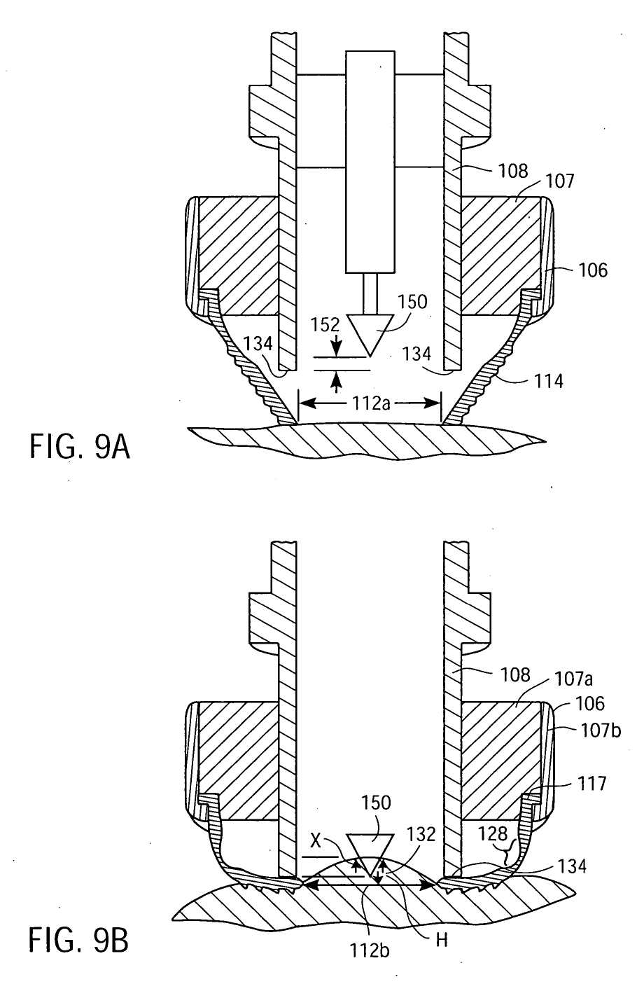

FIGS. 9A and 9B illustrate an example lancet 150 (not to scale). The distance from the tip of the lancet 150 to the stop 134 in the direction of travel of the lancet is labeled 152. By providing a stop 134, which limits the degree of folding of cap body 104, a limit is placed on the range of the sizes of bulge 132 which can be produced within the cap. Thus, for a nominal bulge height H (e.g., a bulge that is 3.7 mm in height), the penetration depth X of lancet 150 into the skin can be estimated. Lip 117 is sandwiched between portion 107a and 107B of retainer 106, as illustrated in FIG. 9B.

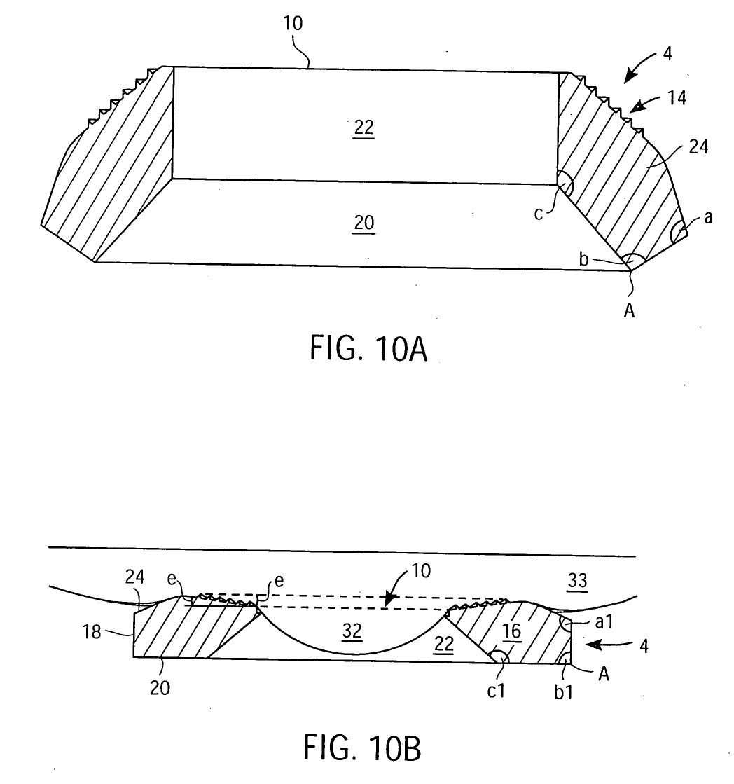

FIGS. 10A and 10B illustrate in more detail the deformation induced in cap body 4 during use. Second internal surface 20 is pushed flat against the base of the retainer ring (not shown) to form a disc shape. In other words, the base portion of the cap body 4 splays out whilst the upper portion closest to the skin moves radially inwards. Indeed, this radially inward motion and rotation of the cap body about pivot A can, for certain cross-sectional shapes of cap body 4, cause the formation of a recess immediately adjacent bulge 32. Thus, the portion of the cap body surface containing dermal tissue engaging features 14 is now at an angle “e” to a plane containing opening 10.

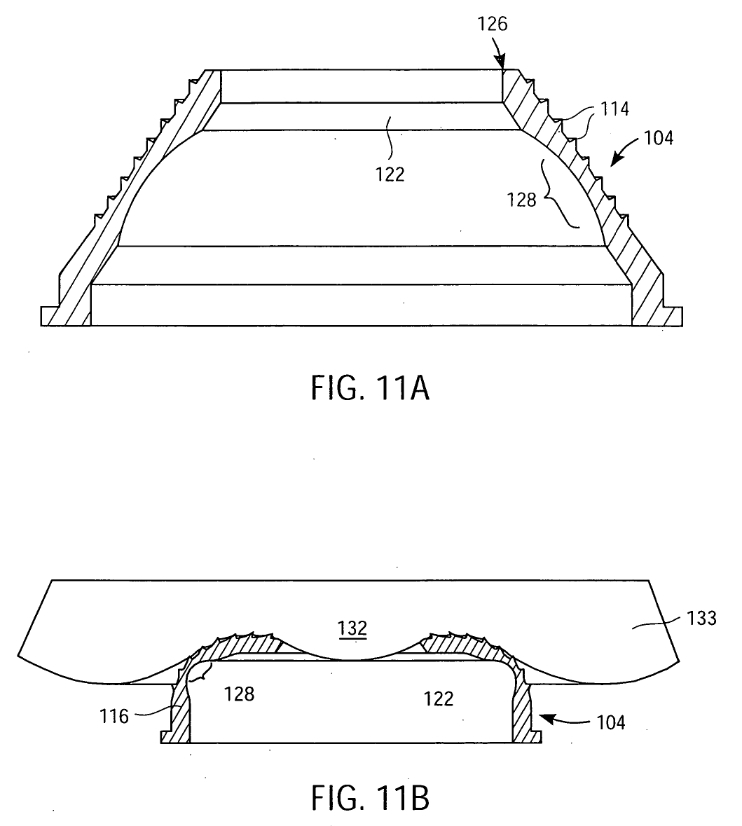

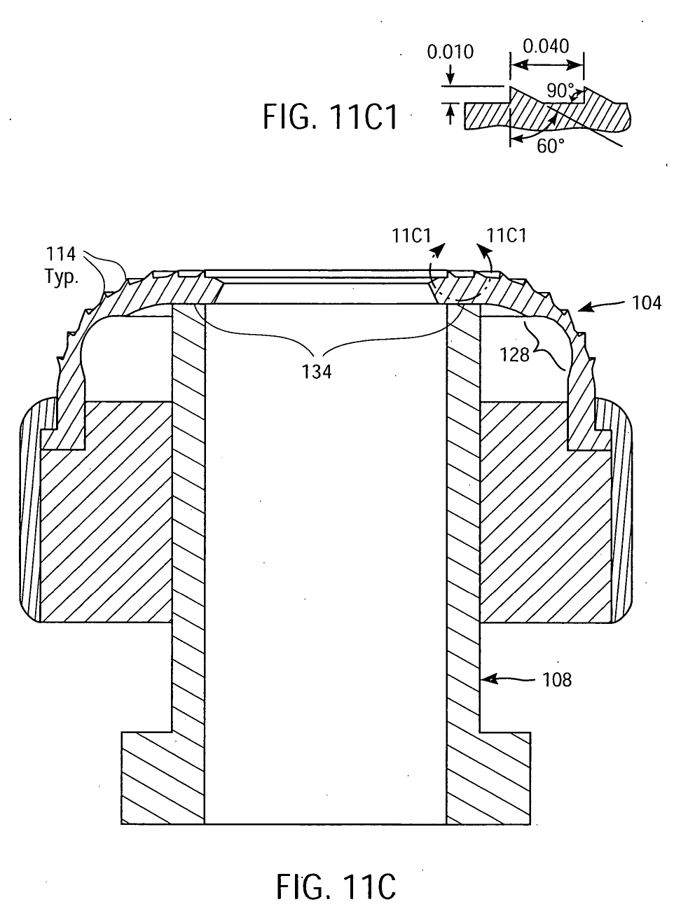

FIGS. 11A and 11B illustrate in more detail the folding over of alternative cap body 104. In addition, these figures show the increased deformation of the cap body in the region of area of weakness 128. FIG. 11C illustrates the shape of dermal tissue engaging features 114 in more detail and includes an exploded cross-sectional illustration of a two concentric protruding ridges.

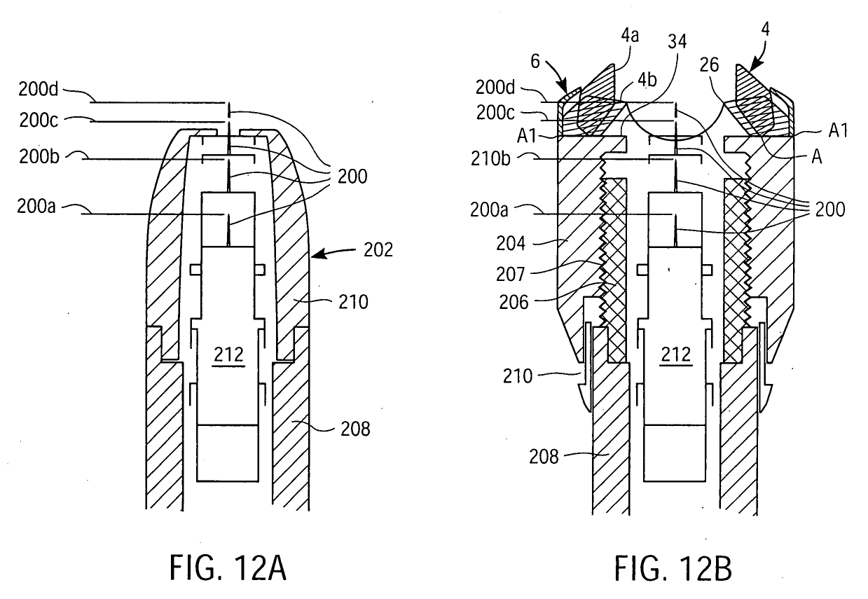

FIGS. 12A and 12B illustrate a conventional lancing device and a lancing device according to the present invention, respectively. In FIG. 12A, a conventional lancing device 202 is shown having a rear housing portion 208 and front housing portion 210 within which a lancet mechanism 212 is mounted. A needle 200 is mounted to the lancing mechanism and is shown in four positions: a rest position 200b, a cocked ready-for-lancing position 200a, a minimum lanced needle depth into the skin position 200c and a maximum lanced needle depth into the skin position 200d. Positions 200c and 200d can be, for example, 1.8 mm apart, the actual lancing position of the needle within this range is determined by a click-mechanism situated at the far end of rear housing portion 208 (not shown).

FIG. 12B shows a modified lancing device according to one aspect of the present invention incorporating a further adjustment mechanism with two co-operating threaded portions 204 and 206 (threaded at 207). Adjustment of these threads allows the position of stop 34 (and hence inner edge 26 of cap body 4 when compressed) to be controlled relative to the needle positions 200a, 200b, 200d and 200d as set by the click mechanism on the far end of the housing. The net result of these two adjustments (threads 207 and the click mechanism) determines needle depth into the skin. An alternative implementation would be to have just one of these two adjustments (i.e., one of threads 207 and the click mechanism), the effect of which can be, for example, a total range of approximately 5 mm. Table 5 below shows the net result of these two adjustments on the needle travel into the skin for a typical bulge of the skin past inner edge 26 of 3.7 mm.

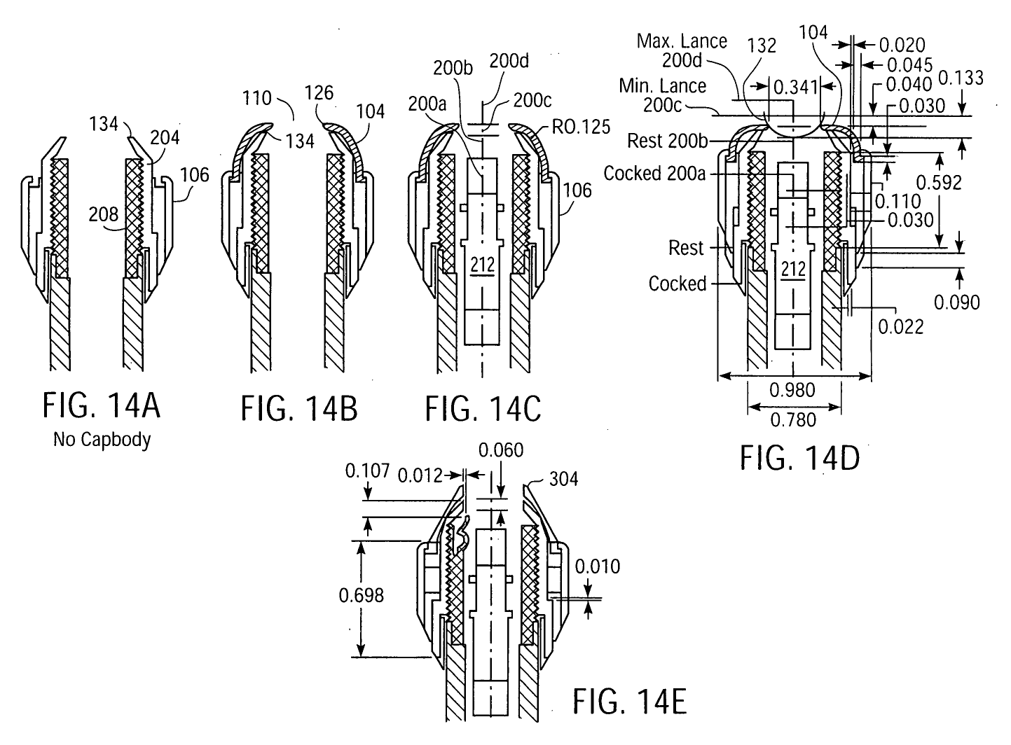

FIG. 14A illustrates a lancing device according to one exemplary embodiment of the present invention having a housing including a threaded portion 204 threadedly mounted to another threaded portion 206 to provide longitudinal adjustment of stop 134 (with respect to the direction of lancet travel—not shown). FIG. 14B illustrates cap body 104 in a compressed position. FIG. 14C illustrates the device of FIGS. 14A and 14B, now including a lancet mechanism 212, and showing needle positions 200a (a cocked position ready for lancing), 200b (an at rest position), 200c (a minimum lanced position) and 200d (a maximum lanced position as adjusted by the click mechanism). FIG. 14D illustrates a lancing device in which cap body 104 is wholly compressed and the bulge 132 has been formed in the dermal tissue. FIG. 14E illustrates the lancing device of FIG. 14D with cap body 304 in a relaxed position.

### EXAMPLE 1

Comparison of Performance of a Conventional Rigid Cap with a Cap According to the Present Invention (referred to as a “Soft Cap”):

A comparative study between a conventional rigid cap (i.e., rigid cap #1) and a cap according to the present invention (referred to as a “Soft Cap”) was conducted using a 30 gauge lancet (i.e., an Ultra-Fine II lancet™) available from Beckton Dickinson of Franklin Lakes, N.J. As can be seen from the results listed in Table 1, the average amount of blood produced in the comparative study was significantly greater using a Soft Cap than the conventional cap. The minimum amount of blood produced using a Soft Cap was 0.8 μl, an amount that was significantly greater than that produced by the conventional rigid cap. The overall success rate in terms of the percentage number of lancing events giving greater than 1 μl of blood (typically the minimum volume required to give an accurate assessment of an analyte, such as glucose, in blood) is 92% for the Soft Cap compared to 25% with the conventional rigid cap.

TABLE 1
BLOOD VOLUME (μl)

| | Average | Minimum | Maximum | Success Rate = % Number | |
|---|---|---|---|---|---|
| Cap | (μl) | (μl) | (μl) | of sticks giving >1 μl blood | |
| Rigid | 0.5 | 0.1 | 1.3 | 25% | BD |
| Cap #1 | | | | | |
| Soft Cap | 4.3 | 0.8 | 6.0 | 92% | Lancet |

Table 2 below illustrates the results of a comparative study that was conducted in a similar manner to that described above bit that employed an alternative conventional lancet with a second type of conventional rigid cap (i.e., rigid cap #2).

TABLE 2
BLOOD VOLUME (μl)

| | Average | Minimum | Maximum | Success Rate = % Number |
|---|---|---|---|---|
| Cap | (μl) | (μl) | (μl) | of sticks giving >1 μl blood |
| Rigid cap | 0.4 | 0.1 | 0.7 | 0% |
| #2 | | | | |
| Soft Cap | 3.3 | 1.3 | 6.3 | 100 |

### EXAMPLE 2

Comparison of Performance with Different Embodiments of the Cap Design

In this study, the embodiment shown in FIGS. 1-4 (L1 in the Table 3) is compared to the embodiment shown in FIGS. 5A to 8 (L2 in Table 3). Users representing extremes were tested: User 1 had cold hands, making it difficult to obtain a blood sample, while User 2 had warm hands making it easier to obtain a sample.

The method comprised pressing the cap body onto the site to be lanced, lancing with a 30-gauge needle, holding the cap body in place for 5 seconds releasing the cap body and collecting blood by a calibrated glass capillary pipette. Multiple opening diameters (before use), needle depth and lancing locations were tested. The needle depth, a higher number corresponds to a greater lance needle depth. Consequently, the greater the lance depth, the more pain the user experiences. In some instances, the thumb was used to aid in obtaining a sample (i.e., some squeeze assist). Success was defined as obtaining more than 1 μl of blood.

The results in Table 3 for User 1 indicate that the design shown in FIGS. 5A to 8 (i.e., design L2) works equally well on the side of the finger or the palm, whereas design L1 does not work as well on the palm. The L1 cap design can also require some squeeze assist when the opening is 13 mm in diameter and is used on the side of the finger at a needle depth of 4.

No thumb squeeze assist was required when cap L2 was used, regardless of needle depth. The results for User 2 with the cap design L2 were also superior to those with cap design L1. As with User 1, the design L2 required thumb squeeze assist in order to obtain sufficient blood from the side of the finger, whereas design L2 did not. These results indicate that design L2 is more versatile and can be used in multiple locations. This may be due to the fact that design L2 collapses in on itself (i.e., it folds inwards) when a certain predetermined force is applied to it.

TABLE 3
Summary of user data with cap designs

| | | | | | | | | | |
|---|---|---|---|---|---|---|---|---|---|
| | Aperture | | | | | | | Thumb | |
| Cap | Diameter | Needle | | | Min | Max | Avg | Squeeze | |
| Design | (mm) | Depth | Location | N | (uL) | (uL) | (uL) | Assist | Success |

User 1 - cold hands

| | | | | | | | | | |
|---|---|---|---|---|---|---|---|---|---|
| L1 | 13 | 4 | side finger | 4 | 0.5 | 1.3 | 1 | N | 3/4 |
| L1 | 13 | 4 | side finger | 3 | 2 | 3.5 | 2.6 | Y | 3/3 |
| L1 | 11 | 4, 1 | side finger | 4 | 2.6 | 32 | 2.9 | N | 4/4 |
| L1 | 11 | 7 | palm | 3 | 0.25 | 0.5 | 0.5 | N | 0/3 |
| L2 | 10 | 7, 4, 1 | palm | 3 | 2.3 | 4 | 3.4 | N | 3/3 |

User 2 - warm hands

| | | | | | | | | | |
|---|---|---|---|---|---|---|---|---|---|
| L1 | 11 | 1 | side finger | 8 | 0.5 | 2 | 1.5 | N | 7/8 |
| L1 | 11 | 1 | side finger | 8 | 1.5 | 4.2 | 2.9 | Y | 8/8 |
| L2 | 10 | 3 | side finger | 8 | 1.1 | 4.5 | 2.8 | N | 8/8 |
| L2 | 10 | 3 | side finger | 8 | 1.5 | 4.8 | 2.9 | Y | 8/8 |
| L2 | 10 | 3 | palm | 8 | 1.3 | 3.8 | 2.9 | N | 8/8 |

### EXAMPLE 3

Study of Caps According to the Present Invention Formed of Various Materials

Four different cap materials were tested with the embodiment shown in FIGS. 1 to 4, (device L1) and the results are shown in table 4. Two types of silicone (of hardness 40 A and 60 A on the Shore Index) and two types of polyurethane (30 A and 50 A in the Shore Index) were used. Blood volume obtained upon lancing was measured and the pain was assessed for 32 participants. Each participant received two finger sticks with each cap design on the same finger of both hands for a total of eight finger sticks per participant. The blood volume was measured with calibrated capillary pipettes and the pain scale is given below table 4.

Success was defined as obtaining more than 1 μl of blood. As is shown in Table 4, blood volume with each type of cap was greater than 1 μl. Pain was barely noticeable with each material. Use of all but the silicone 60A resulted in a success rate greater than 90%. The greatest blood volume, highest pain rating and highest success rate was obtained with the silicone 40A cap. It will thus be appreciated by those skilled in the art that a variety of materials which are resiliently deformable can be used in caps according to the present invention.

TABLE 4
Data Comparing cap Material

| | Average | Average | |
|---|---|---|---|
| | Blood | Pain (0- | Success |
| Cap material | Volume (μl) | 10) | Rate |
| Silicone 60A | 2.77 | 2.24 | 78.13 |
| Polyurethane 30A | 3.25 | 2.47 | 93.75 |
| Silicone 40A | 3.54 | 2.72 | 95.32 |
| Polyurethane 50A | 3.09 | 2.40 | 93.75 |

Pain Scale:
0 = could not feel
2 = barely noticeable
4 = slightly painful
6 = somewhat painful
8 = painful
10 = very painful

### EXAMPLE 4

Study of Lancet Adjustment Versus Needle Depth into the Dermal Tissue

Table 5 contains a graph illustrating the approximate needle depth penetration into the skin in millimeters versus the number of clicks (1-7) providing depth adjustment in a conventional lancing device fitted with a conventional rigid cap. The conventional lancing device employed in collecting the data of Table 5 was a Penlet® Plus lancing device, which is commercially available from LifeScan, Inc. of Milpitas, Calif., USA. Thus, point 404 illustrates that at depth setting 5, lancet penetration into the dermal tissue for the Penlet® Plus is 2 mm.

Table 5 also illustrates the variation in needle penetration (in millimeters) into the dermal tissue versus position of the retainer 6 relative to a fixed point on the lancet housing (for example, threaded portion 206 or rear housing portion 208). These data were collected for device L1 (FIGS. 1-4) at depth adjustment 1-7 (number of clicks) using a silicone Soft Cap (with a Shore hardness of 40 A) and a nominal bulge height of 3.7 mm on the side of the finger. Thus, point 406 illustrates that for 2 rotations of retainer 6 (an adjustment equivalent to moving the Soft Cap and stop 34 two millimeters [2 mm] away from its minimum adjustable position), a penetration depth of 2 mm into the dermal tissue results (when the click position is at position #1). The depth of penetration increases as bulge height increase (e.g., increased bulge height caused by increased hardness of cap body 4). Thus, an increase in cap body hardness increases the amount of force that needs to be applied to deflect the cap from 4A to 4B and, therefore, increases the height of the bulge and the needle depth into the skin for the same click position.

As will be appreciated by those skilled in the art, caps, lancet devices incorporating a cap and combined lancet and metering devices incorporating caps according to the present invention greatly facilitate the production of a fluid sample (e.g., a blood sample) at a puncture (lancing) site without requiring a subsequent squeezing/milking action. This facilitates in-situ testing of a fluid sample by means of a fluid collection device (such as a test strip) that is introduced at the puncture site just after a lancet has been retracted. Such a device allows the user to easily undertake two actions (i.e., placing a device over a suitable portion of dermal tissue and launching the device), thereby simplifying the collection of a sample and rending it more convenient for a user.

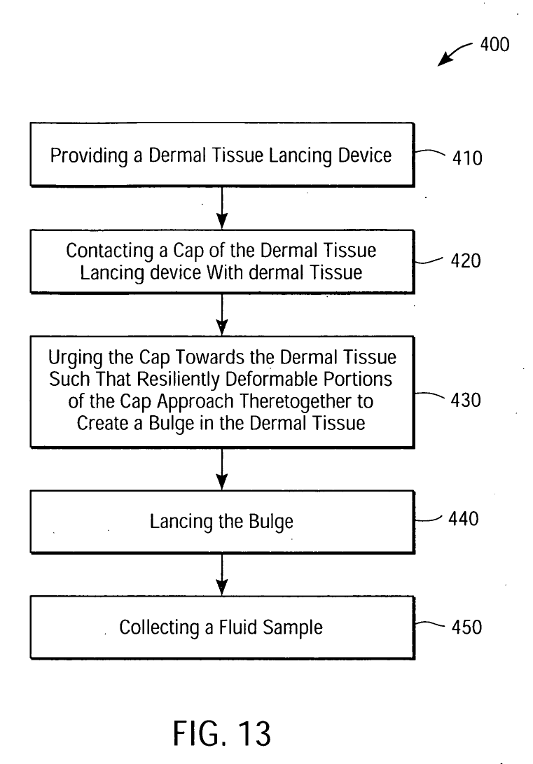

Referring to FIG. 13, a method 400 for the collection of a fluid sample (e.g., a blood sample) from dermal tissue according to an exemplary embodiment of the present invention includes providing a dermal tissue lancing device, as set forth in step 410. The provided dermal tissue lancing device includes a housing, a lancet that is moveable with respect to the housing and a cap. The cap includes a proximal end for engaging with the housing, a distal end for engaging with dermal tissue, and an opening (i.e., aperture) for a portion of the lancet to pass through. The distal end of the cap includes at least first and second resiliently deformable portions for engaging dermal tissue.

Next, as set forth in step 420, the cap of the dermal tissue lancing device is contacted with the dermal tissue (e.g., dermal tissue of a fingertip, limb, abdomen or other site from which a fluid sample is to be collected) such that the at least first and second portions engage the dermal tissue.

The cap is then urged towards the dermal tissue such that the at least first and second portions deform resiliently and approach theretogether, as set forth in step 430. The approaching of the first and second portions, which may or may not occur in a synchronized fashion, creates a bulge in the dermal tissue. The urging together of the first and second portions can create the bulge by, for example, decreasing the size of the dermal tissue lancing device opening. Optionally, the cap can be held for a predetermined time period in such a way that a bulge is maintained in the dermal tissue (i.e., pre-lance pressure).

The bulge is then lanced, using the lancet, to create a puncture in the bulge, as set forth in step 440. Optionally, the cap can be held for a predetermined time period in such a way that a bulge is maintained in the dermal tissue subsequent to the lance (i.e., post-lance pressure). Preferred time duration for the post-lance pressure is approximately 5 seconds. A fluid sample is then collected from the puncture, as set forth in step 450. One skilled in the art will recognize that method 400 can be modified to employ any of cap, lancing device or combined lancing and metering device according to the present invention.

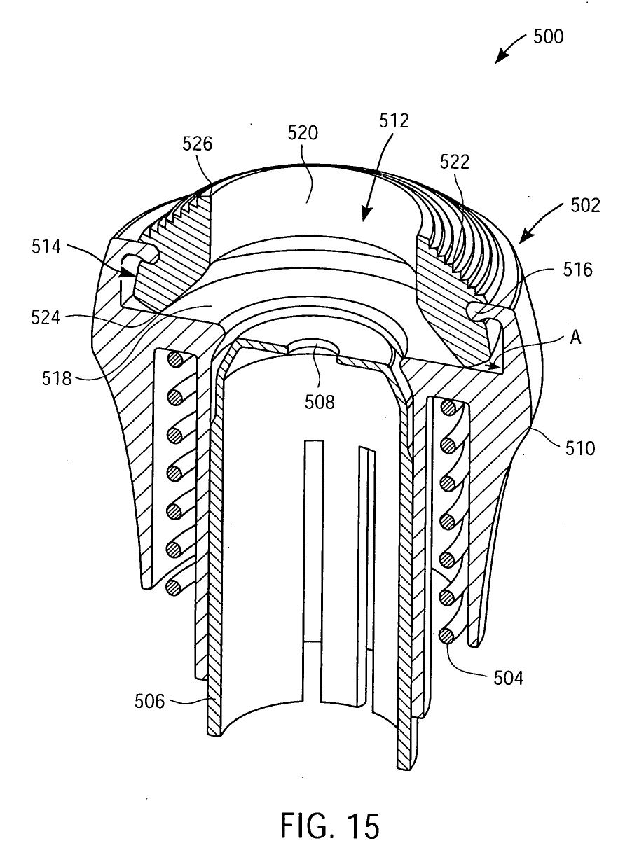

FIG. 15 is a cross-section schematic view of a lancing device 500 according to an exemplary embodiment of the present invention. Lancing device 500 includes a cap 502 (also according to an embodiment of the present invention), a spring 504 and a floating probe 506. Floating probe 506 includes an aperture (i.e., opening) 508 configured for a lancet (not shown) to pass therethrough. Floating probes, such as floating probe 506 of FIG. 15, are described in co-pending U.S. patent application Ser. No. 10/690,083, which is hereby incorporated in full by reference.

Cap 502 includes a retainer 510 and a flexible cap body 512. Retainer 510 has an inwardly facing recess 514 for receiving flexible cap body 512. Retainer 510 also has an inwardly protruding rim 516, and a base surface 518.

Flexible cap body 512 has an opening 520 configured to allow a lancet (not shown) to pass therethrough, an outer surface (rebate) 522, a lower rim 524 and an upper rim 526.

Retainer 510 rests on spring 504, which in turn rests on an upper housing (not shown) of lancing device 500. Inwardly protruding rim 516 of retainer 510 is configured to operatively cooperate (as described below) with outer surface 522 of flexible cap body 512. Lower rim 524 of flexible cap body 512 provides for contact between the flexible cap body 512 and base surface 518 of retainer 510.

During use of lancing device 500, upper rim 526 (also referred to as the distal end) of flexible cap body 512 is pushed against a target site (e.g., dermal tissue) by movement of the lancing device towards the target site, causing flexible main cap body 512 to compress and rotate about protruding rim 516 of retainer 510. Lower rim 524 (also referred to as the proximal end) of flexible cap body 512 slides outwards along base surface 518 of retainer 510. Meanwhile, upper rim 526 grips (or slides then grips) the target site, causing the target site to bulge. During compression, outer surface 522 travels over, and pivots with respect to, protruding rim 516 of retainer 510. In addition, during use, flexible cap body 512 travels within inwardly facing recess 514 in the direction of arrow “A”.

From the foregoing description, one skilled in the art will recognize that flexible cap body 512 of lancing device 500 can be considered as including two portions (i.e., first and second symmetric portions with one of the portions being depicted in FIG. 15) that form a continuous ring adapted for engaging and surrounding dermal tissue.

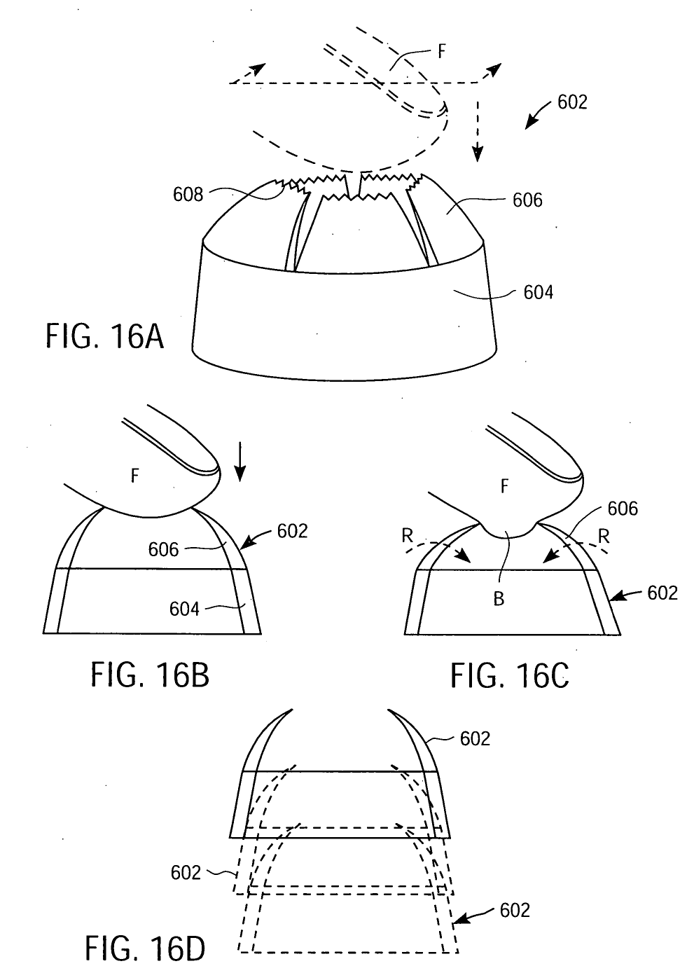

FIGS. 16A through 16D are various views of a cap 602 according to another exemplary embodiment of the present invention. FIG. 16A is a simplified perspective view of cap 602 with a user's finger F shown in dashed lines. Cap 602 includes a main portion 604 (e.g., an injection molded main portion) and a plurality of inwardly bendable portions 606. Each of the inwardly bendable portions includes a plurality of skin-gripping teeth 608. FIG. 16A depicts finger F approaching the plurality of inwardly bendable portions 606.

FIG. 16B depicts finger F touching the plurality of inwardly bendable portions 606 and being urged towards cap 602 in the direction of the arrow. In FIG. 16C, finger F has been urged towards cap 602 such that inwardly bendable portions 606 have bent inwardly in the direction of dashed arrows “R”, causing a skin bulge “B” to form. The formation of skin bulge B is facilitated by the purchase afforded by skin-gripping teeth 608. FIG. 16D illustrates a manner in which the configuration of cap 602 provides for a plurality of caps 602 (two of which are depicted with broken lines) can be stacked to allow easy storage.

During use, there is a potential for caps to embodiments of the present invention to come into contact with blood or other bodily fluid. Such contact can conceivably lead to contamination of the cap with undesirable micro-organisms (e.g., bacteria or fungi). Therefore, it can be beneficial for caps according to embodiments of the present invention to be formed, at least partially, of an anti-microbial material, anti-fungal material and/or anti-viral material, for example, anti-microbial plastic, anti-microbial resin and/or anti-microbial silicone. Suitable anti-microbial materials include anti-microbial compounds that include trichloro-phenol group, such as 2,4,4-trichloro-2-hydroxy diphenol ether. The anti-microbial compound can be, for example, a coating of the cap or incorporated directly in the cap.

### EXAMPLE 5

Theoretical Mechanical Analyses of an Exemplary Cap

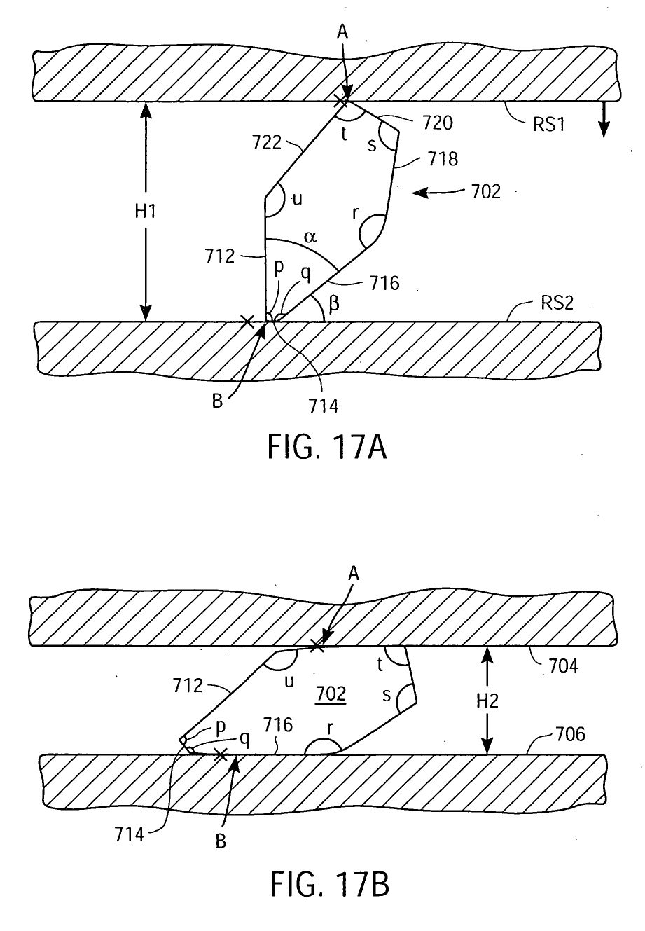

Insight into the use (i.e., compressive operation) of caps according to embodiments of the present invention was obtained through a theoretical mechanical analysis of an exemplary cap configuration. FIGS. 17A and 17B show a cross-section through the cap 702 prior to and after compression, respectively, by opposing rigid surfaces RS1 and RS2. In this analysis, the cross-section of the exemplary cap is six-sided. Initial points of contact between cap 702 and RS1 and RS2 are labeled “A” and “B” respectively. Furthermore, the initial distance between RS1 and RS2 is labeled H1 (see FIG. 17A), while the compressed distance is H2 (see FIG. 17B).

Cap 702 has six sides, 712, 714, 716, 718, 720 and 722. In addition, the internal angles of the cross section of cap 702 are shown as angles p, q, r, s, t and u. Furthermore, the angle between sides 712 and 716 is angle α, while the angle between side 716 and RS2 is angle β. Angle α and angle β determine the final position of cap 702 after compression. For example, a relatively small angle α can result in cap 702 moving further from point B upon compression than would be the case with a relatively large angle α.

In the positioning of FIG. 17B, the geometry of cap 702 prevents further rotation. Any further increase in force will act to compress cap 702 rather than encourage further rotation.

Since caps are generally not compressed between two parallel rigid plates during use, one skilled in the art will recognize the above analysis, and related FIGS. 17A and 17B, is presented for descriptive purposes only.

It should be understood that various alternatives to the embodiments of the invention described herein may be employed in practicing the invention. It is intended that the following claims define the scope of the invention and that methods and structures within the scope of these claims and their equivalents be covered thereby.

That which is claimed is:

1. A cap for a dermal tissue lancing device, the dermal tissue lancing device including a housing and a lancet that is movable with respect to the housing, the cap comprising:
a proximal end for engaging with the housing;
a distal end for engaging with dermal tissue; and
an opening for a portion of the lancet to pass through;
wherein the distal end includes at least a first portion and a second portion for engaging dermal tissue, the at least first and second portions being resiliently deformable such that, when the cap contacts and is urged towards dermal tissue, the at least first and second portions deform resiliently, engage the dermal tissue and approach theretogether.

2. The cap of claim 1 in which at least one of the first portion and second portion includes an edge portion that serves to define a part of the opening.

3. The cap of claim 1, wherein the at least first and second portions are adapted such that when the cap contacts and is urged toward dermal tissue, the at least first and second portions deform resiliently such that the opening is reduced in size.

4. The cap of claim 1 in which the at least first and second portions form a continuous ring adapted for engaging and surrounding dermal tissue.

5. The cap of claim 4, wherein the continuous ring is substantially circular.

6. The cap of claim 1 in which the cap is at least partially formed of a resilient, deformable material.

7. The cap of claim 6, wherein the cap is formed from a resilient deformable material selected from the group consisting of elastomeric materials, polymeric materials, polyurethane materials, latex materials, silicone materials, and combinations thereof.

8. The cap of claim 1, wherein the cap is formed of a material sufficiently transparent to distinguish a fluid sample to be withdrawn with the dermal tissue lancing device.

9. The cap of claim 1 formed of a non-blood colored material.

10. The cap of claim 1, wherein the cap is of a graded resilience such that the distal end is more resilient than the proximal end.

11. The cap of claim 1, wherein the cap is at least partially formed of an anti-microbial material.

12. The cap of claim 11, wherein the anti-microbial material includes a trichloro-phenol compound.

13. The cap of claim 12, wherein the trichloro-phenol compound is 2,4,4-Trichloro-2-hydroxy-diphenol.

14. The cap of claim 1, wherein the cap includes a coating of anti-microbial material.

15. The cap of claim 1, wherein the at least first and second portions are inwardly bendable portions.

16. The cap of claim 15, wherein the inwardly bendable portions include skin-gripping teeth.

17. The cap of claim 1, wherein the at least first and second portions have a six-sided cross-section.

18. The cap of claim 1, wherein the at least first and second portions have a six-sided cross-section, the cross-section including an internal angle that determines a compressed position of the cap during use.

19. The cap of claim 1, wherein the cap is configured to be stackable with other identical caps.

20. The cap of claim 1, wherein the at least first and second portions deform resiliently, engage the dermal tissue and approach theretogether such that a bulge is created in the dermal tissue.

21. The cap of claim 20, wherein the cap is configured for applying a pre-lance pressure to maintain the bulge prior to lancing by the dermal tissue lancing device and for applying a post-lance pressure to further maintaining the bulge after lancing by the dermal tissue lancing device.

22. A cap for a dermal tissue lancing device, the dermal tissue lancing device including a housing and a lancet that is movable with respect to the housing, the cap comprising:
a proximal end for engaging with the housing;
a distal end for engaging with dermal tissue; and
an opening for a portion of the lancet to pass through;
wherein the distal end includes at least a first portion and a second portion for engaging dermal tissue, the at least first and second portions being resiliently deformable such that, when the cap contacts and is urged towards dermal tissue, the at least first and second portions deform resiliently such that the opening is reduced in size, engage the dermal tissue and approach theretogether.

23. A cap for a dermal tissue lancing device, the dermal tissue lancing device including a housing and a lancet that is movable with respect to the housing, the cap comprising:
a cap body;
a retainer; and
an opening for a portion of the lancet to pass through;
wherein the cap body has a proximal end for engaging with the retainer and a distal end for engaging with dermal tissue; and
wherein the retainer has a proximal end for engaging with the housing and a distal end for engaging with the cap body; and
wherein the distal end of the cap body includes at least a first portion and a second portion for engaging dermal tissue, the at least first and second portions being resiliently deformable such that, when the cap contacts and is urged towards dermal tissue, the at least first and second portions deform resiliently, engage the dermal tissue and approach theretogether.

24. The cap of claim 23, wherein the proximal end of the cap body is prevented by the retainer from deforming outwardly in a plane substantially parallel to a plane containing the opening.

25. The cap of claim 23, wherein the proximal end of the cap body freely rests within the retainer.

26. The cap of claim 23, wherein the proximal end of the cap body is fixedly mounted to the retainer.

27. The cap of claim 23, wherein the retainer includes a stop for preventing deformation of the cap body in a direction generally perpendicular to a plane containing the opening.

28. The cap of claim 23, wherein a cross-sectional shape of the cap is such that a pivot is formed on the cap body and within the retainer such that when the cap, including the cap body and retainer, is urged towards dermal tissue the cap body pivots within the retainer such that the size of the opening is reduced.

29. The cap of claim 23, wherein a cross-sectional shape of the cap body is such that when the cap is urged towards the dermal tissue of a user, the cap body experiences one of falling and folding in on itself once a predetermined urging force is applied, thereby reducing the size of the opening.

30. The cap of claim 29, wherein the cap body includes at least one weak region so as to enable the cap body to fold in on itself once the predetermined force is applied.

31. The cap of claim 23, wherein the cap body and retainer are formed as an integral unit.

32. The cap of claim 23, wherein the size of the opening is reduced once the cap has been urged towards dermal tissue.

33. The cap of claim 23, wherein the retainer has an inwardly facing recess for receiving the cap body and an inwardly protruding rim configured to operatively cooperate with an the outer surface of the cap body.

34. The cap of claim 23, wherein the cap body is a flexible cap body.

35. A lancet device comprising:
a housing;
a lancet that is moveable with respect to the housing; and
a cap that includes:
a proximal end for engaging with the housing;
a distal end for engaging with dermal tissue; and
an opening for a portion of the lancet to pass through;
wherein the distal end includes at least a first portion and a second portion for engaging dermal tissue, the at least first and second portions being resiliently deformable such that, when the cap contacts and is urged towards dermal tissue, the at least first and second portions deform resiliently, engage the dermal tissue and approach theretogether.

36. The lancet device of claim 35, wherein the cap includes:
a flexible cap body; and
a retainer.

37. A combined lancet and metering device for lancing dermal tissue, extracting a fluid sample and measuring an analyte within said fluid sample, the combined lancet and metering device comprising:
a housing;
a lancet that is moveable with respect to the housing;
a cap that includes:
a proximal end for engaging with the housing;
a distal end for engaging with dermal tissue; and
an opening for a portion of the lancet to pass through;
wherein the distal end includes at least a first portion and a second portion for engaging dermal tissue, the at least first and second portions being resiliently deformable such that, when the cap contacts and is urged towards dermal tissue, the at least first and second portions deform resiliently, engage the dermal tissue and approach theretogether; and
a metering device for measuring the analyte within the fluid sample.

38. A combined lancet and metering device for lancing dermal tissue, extracting a fluid sample and measuring an analyte within the fluid sample in which a fluid collection device is delivered to a position adjacent a lancing position after lancing has occurred such that the fluid sample can be collected and measured within the metering device without repositioning of the combined lancet and metering device with respect to the puncture, the combined lancet and metering device comprising:
a housing;
a lancet that is moveable with respect to the housing; and
a cap that includes:
a proximal end for engaging with the housing;
a distal end for engaging with dermal tissue; and
an opening for a portion of the lancet to pass through;
wherein the distal end includes at least a first portion and a second portion for engaging dermal tissue, the at least first and second portions being resiliently deformable such that, when the cap contacts and is urged towards dermal tissue, the at least first and second portions deform resiliently, engage the dermal tissue and approach theretogether.

39. The combined lancet and metering device of claim 38, wherein the fluid collection device is a test strip.

40. A method for the collection of a fluid sample from dermal tissue, the method comprising:
providing a dermal tissue lancing device that includes:
a housing;
a lancet that is moveable with respect to the housing; and
a cap that includes:
a proximal end for engaging with the housing;
a distal end for engaging with dermal tissue; and
an opening for a portion of the lancet to pass through;
wherein the distal end includes at least a first portion and a second portion for engaging dermal tissue, the at least first and second portions being resiliently deformable;
contacting the cap with the dermal tissue such that the at least first and second portions engage the dermal tissue;
urging the cap towards the dermal tissue such that the at least first and second portions deform resiliently and approach theretogether, thereby creating a bulge in the dermal tissue;
lancing the bulge using the lancet to create a puncture in the bulge; and
collecting a fluid sample from the puncture.

41. The method of claim 40, wherein the urging step decreases the size of the opening due to the approaching theretogether of the at least first and second portions.

42. The method of claim 40, wherein the collecting step includes collecting a blood sample from the puncture.

43. The method of claim 40 further including:
applying pre-lance pressure to the dermal tissue by holding the cap against the dermal tissue following the urging step and prior to the lancing step, thereby maintaining the bulge.

44. The method of claim 40 further including: and
applying a post-lance pressure to the dermal tissue by holding the cap against the dermal tissue following the lancing step, thereby further maintaining the bulge.

45. The method of claim 44, wherein the applying of the post-lance pressure occurs for a predetermined time of approximately 5 seconds.

46. A method for the collection of a fluid sample from dermal tissue, the method comprising:
providing a dermal tissue lancing device that includes:
a housing;
a lancet that is moveable with respect to the housing; and
a cap that includes:
a proximal end for engaging with the housing;
a distal end for engaging with dermal tissue; and
an opening for a portion of the lancet to pass through;
wherein the distal end includes at least a first portion and a second portion for engaging dermal tissue, the at least first and second portions being resiliently deformable;
contacting the cap with the dermal tissue such that the at least first and second portions engage the dermal tissue;
urging the cap towards the dermal tissue such that the at least first and second portions deform resiliently and approach theretogether, thereby creating a bulge in the dermal tissue;
lancing the bulge using the lancet to create a puncture in the bulge;
applying a post-lance pressure to the dermal tissue by holding the cap against the dermal tissue, thereby further maintaining the bulge; and
collecting a fluid sample from the puncture.
